(19) United States
(12) Patent Application Publication
(10) Pub. No.: US 2015/0374451 A1
(43) Pub. Date: Dec. 31, 2015

(54) MESOSCOPIC TUMOR MICROENVIRONMENT IMAGING WITH IMPROVED CONTRAST

(71) Applicant: Purdue Research Foundation, West Lafayette, IN (US)

(72) Inventor: Young L. Kim, West Lafayette, IN (US)

(73) Assignee: PURDUE RESEARCH FOUNDATION, West Lafayette, IN (US)

(21) Appl. No.: 14/735,102

(22) Filed: Jun. 9, 2015

Related U.S. Application Data
(63) Continuation-in-part of application No. 13/964,722, filed on Aug. 12, 2013.
(60) Provisional application No. 62/009,724, filed on Jun. 9, 2014.

(51) Int. Cl. A61B 19/00; A61B 5/00; A61B 1/00; A61B 1/05; A61B 1/06

(52) U.S. Cl. CPC A61B 19/5244; A61B 1/051; A61B 1/0646; A61B 1/00009; A61B 5/0084; A61B 2019/5257; A61B 2019/5265; A61B 2562/028

(57) ABSTRACT

A mesoscopic imaging and surgical guidance apparatus. The apparatus includes an image capturing path, including a telecentric lens which receives backscattered light from a light which illuminates tissue in vivo with diffused light or collimated light and transforms the received light into an orthographic view, an image capturing device which captures the orthographic view, a processing unit configured to process the captured orthographic view to thereby generate a guidance image, and a projection path, including a light source configured to project the generated guidance image through the telecentric lens on to the tissue to guide surgical operations.

This application is a continuation in part application of U.S. application Ser. No. 13/964,722 filed Aug. 12, 2013, and claims the benefit of U.S. provisional application having Ser. No. 62/009,724 filed Jun. 9, 2014, each of which is incorporated by reference in its entirety.

This invention was made with government support under ES020965, AR053710, TR000162, and R03 CA153982 awarded by National Institutes of Health. The government has certain rights in the invention.

### BACKGROUND OF THE INVENTION

A. Field of the Invention

The invention relates generally to an apparatus and method of mesoscopic tumor imaging which provide improved tumor contrast.

B. Description of the Related Art

Advances in cancer imaging rely on the development of methods or probes that can provide unique contrasts for tumors and tissue microenvironments. Numerous studies have shown that morphological, structural, and organizational changes in tissue can play an important role in tumor initiation and progression in several types of cancer.

Various methods of optical gating have been used in biomedical imaging, including coherence gating, time gating and spatial gating, each of which has its strengths and weaknesses. Light elastically scattered from scattering media including biological tissue can be classified into three major components: ballistic light, snake-like light, and diffuse light. The ballistic or snake-like components are usually masked by the multiple scattered diffuse component, which generates complicated scattering paths and deteriorates contrast in images. Imaging of light backscattered from biological tissue has recently received considerable attention to probe structural alternations at subcellular levels.

Recent advances in deep tissue imaging are based on the separation of the ballistic or snake-like light from the diffuse light to improve the image contrast. Among various approaches, the simplest method to select the ballistic or snake-like light is directional gating (or spatial filtering), because it propagates along the direction of the incident light, whereas the diffuse light exits the medium at oblique angles with respect to the optical axis.

In order to determine whether the anisotropy factor of a scattering medium plays a role in the image formation of an embedded object, Xu et al. studied diffuse light suppression of back-directional gating imaging of contrast targets embedded in aqueous suspensions which contained different sizes of polystyrene microspheres to vary the anisotropy of the medium. J. Biomed. Opt. 14(3), 030510 (Jun. 25, 2009), the contents of which are incorporated herein in their entirety by reference. The contrast target was placed at different optical thicknesses below the sample surface. The results showed the potential for contrast improvement in high anisotropic media.

### SUMMARY OF THE INVENTION

There is a trade-off in optical imaging between field of view and resolution and sensitivity. Microscopic imaging provides high resolution and sensitivity, but a reduced field of view. Macroscopic imaging provides a wide field of view, but low resolution and sensitivity. The present invention balances high resolution and sensitivity with field of view and used mesoscopic imaging to image tumors.

Thus, the present invention provides a method of imaging tissue on a mesoscopic scale, comprising illuminating tissue in vivo with light, varying at least one parameter of the light with a spectrometer, such as a tunable filter, and forming an image of the tissue by collecting backscattered light from the tissue using any one of (i) a small aperture 4-focal length (4-f) lens system within an angular cone of 2°-5°, (ii) a telecentric lens, and (iii) an anti-scatter grid and a camera lens. Each of (i), (ii) and (iii) optics employs back-directional gating. In one embodiment, images of the tissue are formed via back directional gating with a small aperture 4-focal length (4-f) lens system within an angular cone of 2°-5°, and preferably a solid angle of θ=±2° in the exact backward direction is used. As used herein, a small aperture 4-f lens refers to a lens have an aperture size of about 0.5 to 2.0 mm. In another embodiment, images of the tissue are formed via a telecentric lens, which may be an inline telecentric lens. In a further embodiment, images of the tissue are formed using an anti-scatter grid and a camera lens.

The method of the invention can image an area of tissue at least 10 mm in diameter and up to 100 mm in diameter as a single image. Imaging depth is as deep as 2 mm from the surface.

The method according to invention can additionally comprise determining whether the tissue is neoplastic or pre-neoplastic. In one embodiment the tissue imaged is skin, and areas of tissue with basal cell carcinoma, squamous cell carcinoma and/or melanoma are identified.

The method can further comprise analyzing the image to determine organization and orientation of extracellular matrix in the tissue. In one embodiment, the image is analyzed to determine subclinical hyperemia. In another embodiment, the image is analyzed to determine microvascular hemoglobin content. In either case, the analyzing can comprise comparing data from the image to data stored in a database, wherein the data in the database correlates to degrees of subclinical hyperemia in tissues or the microvascular hemoglobin content. These methods additionally can comprise predicting, based on the analyzing, an area of the tissue at risk for development of skin cancer.

One embodiment of the invention is a method additionally comprising determining whether or not the tissue is damaged by radiation and, if it is damaged, identifying the location of radiation-induced damage. In a further embodiment, the method additionally comprises identifying areas of the tissue for chemopreventative or prophylactic therapies to prevent neoplasia.

In another embodiment, the method additionally comprises demarcating tumor margins for a surgeon in real time during surgery.

The present invention further provides a mesoscopic imaging apparatus, comprising a light which illuminates tissue in vivo, a spectrometer which varies at least one parameter of the light, and optics which transmit light backscattered from the tissue, the optics comprising any one of (i) a small aperture 4-focal length (4-f) lens system within an angular cone of 2°-5°, (ii) a telecentric lens, and (iii) an anti-scatter grid and a camera lens. The mesoscopic imaging apparatus additionally may comprise a camera which forms an image with the light transmitted by the optics and/or a processor connected to the spectrometer and to the camera, wherein the processor controls the variation of the parameter by the spectrometer and receives and stores the image formed by the camera.

The mesoscopic imaging apparatus additionally may include a laser-guided implement which is controlled by the processor. The laser-guided implement may be a pen which draws an outline on the tissue based on a signal transmitted by the processor, the outline being determined by the image stored in the processor, or a treating instrument which treats the tissue based on a signal transmitted by the processor. Such treating may comprise resection of the tissue.

In one embodiment, the processor stores data which correlates image appearance to hyperemia of tissue. In some cases, the processor stores hyperemia data for tissue which is identified as being from an individual patient, and may store hyperemia data for tissue obtained from an individual patient obtained on more than one date.

### BRIEF DESCRIPTION OF THE DRAWINGS

The foregoing advantages and features of the invention will become apparent upon reference to the following detailed description and the accompanying drawings, of which:

FIG. 1 is an imaging system according to a first embodiment.

FIG. 2 is a clinical imaging system according to a second embodiment.

FIG. 3 is a clinical imaging system according to a third embodiment.

FIG. 4 is a clinical imaging system according to a fourth embodiment.

FIG. 5 is a clinical imaging system according to a fifth embodiment.

FIG. 6 is a clinical imaging system according to a sixth embodiment.

FIGS. 7A and 7B show dependence of number of collected photons on the scattering mean free path length for a given anisotropy factor g at two different back-directional angles.

FIGS. 8A and 8B show dependence of diffuse light suppression on anisotropy factor-weighted imaging at different F-numbers of telecentric lenses by calculating resolving power of an object embedded in scattering media.

FIG. 9 is a series showing UVB-induced hyperemic foci in albino mice.

FIGS. 10A and 10B are graphs showing that expanding areas of high Hgb content following cessation of carcinogenic UVB treatment and celecoxib-resistant hyperemic foci exhibit a decrease in the area of high hyperemia.

FIGS. 11(a)-11(b) are telecentric arrangements employing tunable wavelength filters (i.e., an automatic wavelength selection filter) (e.g., mechanical filter wheel, imaging spectrograph, a liquid crystal tunable filter (LCTF)) that can be used to project display of histopathological information on a treatment field.

FIG. 12 is a photograph of an image of a set of (hyper)spectral data from a subject obtained using a telecentric imaging system of a hyperemic area that is and is projected using a digital light projection (DLP) projector including digital micromirror devices (DMD) where the projection beam is coupled to the co-axial port of the telecentric lens using co-registered freckles for providing precise mapping on the treatment field.

FIGS. 13(a)-13(b) are telecentric arrangements without tunable wavelength filters using a 3-color camera (Red-Green-Blue) and an algorithm to provide spectral reconstruction with RGB data.

### DETAILED DESCRIPTION OF SPECIFIC EMBODIMENTS

The inventors surprisingly have discovered that image formation in tissues which uses optics employing back-directional gating removes complicated scattering paths that would otherwise deteriorate the contrast in images. One of the unique optical properties of biological tissue is the tendency that light is scattered in the same direction with respect to the incident direction, and the inventors recognized that this property could be utilized to image tissues, and more particularly epithelial tissue, with high contrast over a wide field of view.

The anisotropy factor g, which is defined as the average cosine of the scattering angle, is a measure of the directionality of elastic light scattering. Thus, the arrangement of aligned or ordered microstructures, e.g., collagen extracellular matrix, dominantly attributes to this unique property. In other words, g can be used as a measure of the cellular and tissue organizations as an intrinsic imaging contrast.

The inventors have discovered that back-directional gated imaging allows g to dominantly attribute to the intensity of light backscattered from tissue, independent of scattering coefficients, and that the g-weighted imaging method can provide a novel contrast for tumors in a mesoscopic, i.e., between microscopic and macroscopic, imaging setup. Back-directional gating can enhance contrast, resolution, imaging depth and imaging area. Unexpectedly, the optics configuration according to the present invention removes unwanted diffusive light because tissue is a highly anisotropic diffusive medium. Another key advantage of g-weighted imaging is a large imaging area, compared with conventional microscopy methods. When tumors are heterogeneous in a large tissue area, e.g., 10 mm×10 mm, examining a small area using microscopic techniques can fail to provide a representative or accurate assessment, requiring a mesoscopic imaging approach.

The use of back-directional gating according to the invention allows the use of scattering anisotropy as an imaging contrast. Importantly, as g increases, the difference in the signal level becomes more significant. A small change at a high level of g can produce a drastic change in the contrast. Given that most biological tissue is highly anisotropic, this indicates the ability to visualize a subtle change in g induced by alterations in tissue structures and organizations. Thus, the scattering intensity or image obtained under back-directional gating is referred to as a g-weighted signal or image.

Neoplastic transformation, i.e., conversion of a normal tissue into a malignant tumor, is highly linked to changes in the extracellular matrix in the stromal tissue, e.g., collagen matrix remodeling and realignment. Several recent methods of using microscopy techniques, such as multiphoton or second harmonic generation microscopy, have shown that morphological and organizational alterations in the extracellular matrix occur in tumor initiation and progression in several types of cancer. Collagen fibers are often aligned and thickened during tumor progression, and this is known as tumor-associated collagen signature. Carcinoma-associated fibroblasts also contribute. Microstructural alterations in the extracellular matrix have been shown in several types of cancer, including skin cancer, breast cancer, and ovarian cancer. Such quantification has also been used to provide a biomarker of prognosis for breast cancer. Although microscopy methods are highly valuable, a large number of microscopic images are required. The present invention covers an equivalent imaging area with just one image.

The present mesoscopic imaging method can be used for intraoperative surgical and histopathological imaging guidance, and allows for detailed tumor visualization in a relatively large area as follows. Because tumors are often heterogeneous in a large tissue area, e.g., 10²-20² mm², examining a small area using microscopic techniques can fail to provide a representative assessment. For example, microscopy provides a relatively small field of view of approximately 0.5² mm², and thus requires that approximately 2,500 microscopy images be stitched together to produce and image area of only 25² mm². This stitching process requires an extreme amount of computational time and power. On the other hand, in simple macroscopic imaging in which the field of view typically is about 100² mm², image contrast and resolution of detailed tumor margins are significantly deteriorated by unwanted diffusive light or crosstalk among adjacent pixels, when minimizing the removal of uninvolved tissue. Thus, there is a definite need for a novel imaging approach to fill this gap.

In tumor-specific endogenous imaging it is mandatory to avoid any use of exogenous contrast agents that can be prone to artifacts due to preparation protocols, and also are time-consuming. Although autofluorescence signals from key endogenous molecules have been intensively studied in several different types of cancer, they are extremely challenging due to photobleaching and low signal-to-noise ratio (SNR). Conventional methods, such as histology, staining microscopy, and biochemical assays, are destructive, time-consuming, expensive, and prone to artifacts due to preparation protocols. Specimen deterioration is also a critical issue for postoperative clinicopathological assessment. In this respect, the present apparatus and method utilizes intrinsic properties of tumors in specific malignancies for intraoperative tumor margin assessment.

The method and apparatus of the invention can identify tumors and, because of the wide field of view and high contrast capability can be used to accurately visualize and demarcate tumor margins. Thus method and apparatus can eliminate the need for Mohs surgery. Mohs surgery is microscopically controlled surgery used to treat common types of skin cancer. During the surgery, after each removal of tissue, while the patient waits, the pathologist examines the tissue specimen for cancer cells, and that examination informs the surgeon where to remove tissue next. The surgeon performing the procedure is also the pathologist reading the specimen slides, and there are limited numbers of such qualified surgeons, and thus skin cancer surgery typically involves travel to a larger hospital which has such a surgeon on staff. Mohs surgery allows for the removal of a skin cancer with very narrow surgical margin and a high cure rate. The present invention would eliminate the need for such specialized surgical expertise, enabling skin cancer surgery to be performed in smaller hospitals and in physician's clinics.

The present method and apparatus also can be used for imaging to identify areas at risk for tumor development, which then can be treated prophylatically in order to prevent, delay and/or minimize tumor development. Inflammation is frequently observed in the tumor microenvironment. Environmental exposures responsible for 90% of cancers exhibit the common feature of promoting inflammation. Studies demonstrate that increased angiogenesis occurs in response to tumor development, and in some cases occurs as a major feature of the transition from premalignancy to invasive cancers. The role of inflammation and angiogenesis in early tumor development may be under-appreciated due to the inability to determine a priori an exact site where a tumor is likely to occur. Microenvironmental changes necessary for tumor development would likely be missed or their significance underestimated if they are non-homogenous in distribution, represent a minor fraction of the available surface, or are not clinically apparent. This is particularly important given that the inflammatory microenvironment that leads to or promotes neoplastic development may differ substantially from the inflammatory microenvironment observed in established tumors.

Recently, probe-based optical assessments of superficial hemoglobin (Hgb) content have shown that a measurable early increase in blood supply is detected not only in the tumor stromal environment, but also is seen as an early change in the surrounding mucosa of the GI tract. Thus, it has been proposed that hyperemia is a feature of field cancerization and is predictive of whether tumors will form. Earlier studies utilizing painstaking microscopic examination of tissues indicated that a pro-angiogenic switch occurs relatively early in tumor development. These studies fail to determine whether hyperemia serves to promote tumorigenesis or results as a generalized response to proangiogenic signals released by hyperplastic or preneoplastic epithelium. Prior to the present invention, it has not been to achieve simultaneous visualization of spatial and temporal alterations in hyperemia to quantitatively analyze and accurately predict sites of tumorigenesis. Such visualization requires a mesoscopic (between microscopic and macroscopic) imaging approach, and the present invention meets the need for such an approach.

The inability specifically to identify tissue sites at the earliest stages of tumorigenesis limits the ability to examine why a risk for tumor development persists. Cyclooxygenase 2 (COX-2) inhibitors, such as celecoxib, are known to suppress UVB-induced inflammation and to suppress photocarcinogenesis. Similarly, COX-2 inhibitors also suppress non-melanoma skin cancer development in humans with significant premalignant actinic disease. The ability of celecoxib to suppress ultraviolet B (UVB)-induced murine skin cancer development after cessation of UVB treatments suggests a potential role for continued subclinical inflammation as a contributing factor in this continued risk. It remains unclear whether the chemopreventive activity of COX-2 inhibitors are chiefly attributable to their anti-inflammatory effects given that COX-2 inhibitors also exhibit potent effects on the growth and survival of normal and neoplastic epidermal keratinocytes.

The present method and apparatus can be used as an adjuvant to Mohs micrographic skin surgery. Non-melanoma skin cancer (NMSC) is the most common and fastest growing type of cancer in the United States (˜1.7 million new cases per year) and is being considered a worldwide epidemic. After traditional treatment, recurrence of the tumors (˜20%) causes increased health care costs, poor cosmesis, complications, and patient anxiety. Among various treatments available for NMSC, Mohs micrographic surgery by Mohs surgeons has the highest rate of complete removal of tumors with the lowest recurrence rate, compared with other treatments performed by dermatologists, ENT surgeons, and plastic surgeons. This is mainly because Mohs micrographic surgery involves microscopic examination of the entire margin of the tumor excision. In this respect, there is a strong need for an intraoperative imaging method for NMSC tumor margin assessment: 1) The delivery and effectiveness of Mohs micrographic surgery have to be enhanced, because it is a tedious and lengthy procedure requiring several unpredictable number of stages. 2) It is critical for other physicians performing treatment of NMSC to have such an imaging system to enhance the effectiveness of other treatment modalities.

Another application for the present method and apparatus is in lumpectomy guidance. Breast cancer is the most frequently diagnosed malignancy in women in the United States (192,370 new cases in 2009). Thanks to progress in screening and early detection, most breast tumors are detected when they are localized and thus lumpectomy with the addition of local radiation is the “standard of care”. Survivals after lumpectomy are shown to be equivalent to those after mastectomy. Despite comparable survival rates of lumpectomy, one critical limitation is that patients receiving lumpectomy have a lifelong risk of local recurrence. Breast tumors often have ill-defined and irregular margins, which make it difficult to precisely determine the true negative margin during surgery. The tissue removed from the breast is examined by a pathologist and an accurate margin status is available in a few days or a week. When positive margins are present at initial excision, i.e. tumor cells are found at the margin, a surgeon has to perform additional surgery to obtain negative margins, because the status of margins is the most predictive factor in local recurrence. Several studies showed that re-excision for the complete removal of the tumors reduces the risk of local recurrence after lumpectomy. As a result, it is estimated that 30-60% of women who undergo lumpectomy will require additional surgery. Additional surgical procedures are associated with increased health care costs, poor cosmesis, complications, and patient anxiety. In the absence of reliable intraoperative margin assessment, the ability to capitalize on the unique opportunities offered by lumpectomy will continue to be handicapped by the requirement of additional surgery.

Often breast tumors can have poorly defined margins, which may make it difficult to determine exactly where the tumor ends and the unaffected tissue begins. Thus, optical imaging techniques that allow depth-selectivity/optical-sectioning and cell/tissue characterization comparable, or even superior, to histology would be ideal for tumor margin delineation. However, because breast tumor margins are often heterogeneous in a large tissue area, examining a small area can fail to provide a representative assessment. In addition, neoadjuvant chemotherapy and endocrine therapy before lumpectomy can cause extra heterogeneous changes in tumor margins. Thus, one key limitation of the current technologies for lumpectomy guidance is that they only allow sampling of a small fraction of tissue. It is practically impossible to examine the entire area of resected tissue using a high-resolution imaging system. For example, a relatively small field of view (e.g. ˜0.3 mm in diameter in confocal microscopy) may require approximately 10,000 images to be stitched together to cover an area of only 30 mm×30 mm. In addition, confocal microscopy and two-photon microscopy typically require extrinsic dyes that may damage or deteriorate specimens for postoperative clinicopathological assessment. In this respect, large-area optical imaging methods would be necessary for accurate margin assessment in lumpectomy. However, in large-area optical imaging, image contrast and resolution of detailed margins are significantly deteriorated by unwanted diffuse light or crosstalk among adjacent pixels. As a result, local optical properties or internal structure variations are typically averaged out. Overall, there is a definite and imperative need for a novel approach to fill the gap between microscopic imaging and macroscopic imaging.

A further application of the present method apparatus is in guidance for head and neck cancer. The method and apparatus can provide intraoperative guidance to ensure that the tumor margin is accurate in patients undergoing head and neck cancer.

When used in a surgical or treatment setting, the mesoscopic imaging system can identify and store data related to tumor margins. The present invention thus can simultaneously image a tumor on a tissue and then project an outline of the image of the tumor onto the tissue. Where the tissue is skin, this data can be used drawn an outline of the tumor on the skin, and the outline can manually be drawn on the tissue, or it can be drawn by a pen connected to and controlled directly by processor 50. For tissues other skin, the area to be resected can visualized for a surgeon using a pattern of light on the tissue, and the surgeon then can treat the tissue guided by the light pattern. The surgeon can use guided laser or robotic surgical implements such as those in a da Vinci surgical system to treat the portion of skin or tissue that the system has identified as being malignant. The treatment may include resection of the tissue.

A particularly valuable application of the present method and apparatus is in the detection of subclinical actinic keratosis, where it can be used in connection with a plan for chemoprevention. The imaging methods can be used to diagnose subclinical actinic keratosis and identify areas of the tissue for chemopreventative or prophylactic therapies to prevent actinic neoplasia. Unlike point-measurements such as conventional bioassays and probe-based methodologies that have shown the ability to predict whether tumors will form, this methodology offers the ability to also pinpoint where tumors are most likely to occur. This is of fundamental importance in dissecting early events in carcinogenesis by determining how areas of high risk for tumor occurrence differ from areas of low risk for tumor formation. The imaging approach of assessing spatiotemporal alterations in microvascularity has great utility as a non-invasive strategy to map out areas of skin with high and low potential for neoplastic development to guide tissue procurement. For example, it can be used in individuals with photodamaged skin and no visible cutaneous neoplasia to determine the risk for future progression to neoplastic disease. In addition, the methodology can be utilized as a non-invasive method to screen chemopreventive agents and to monitor treatment efficacy. The imaging platform offers the advantage of simple and low cost optical design and rapid (<5 minutes) image analysis for clinical studies.

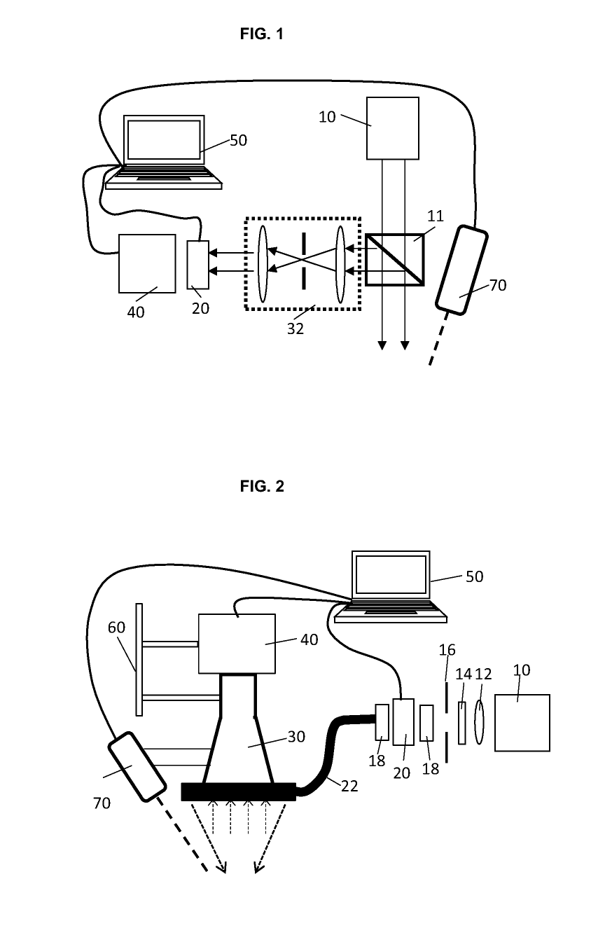

FIG. 1 shows a first embodiment of an optical imaging system of the invention, which has been used in studies in a laboratory setting. It includes broadband light source 10, which may be a xenon lamp, white light LED, or different color LEDs. Light from light source 10 travels to beamsplitter 11 and then to an area of interest or examination (not shown). Reflected light from the tissue goes through back-directional gating component 32, which may be a small aperture 4-focal length (4-f) lens system within an angular cone of 2°-5°. After exiting back-directional gating component 32, the light travels to tunable filter 20, which may be a liquid crystal tunable filter or rotational mechanical filter. Cables connect tunable filter 20 and camera 40 to computer system 50. Camera 40 may be a CCD or CMOS camera, and processor 50 may be a personal computer or laptop.

A second embodiment of an optical imaging system of the invention for use in a clinical setting is shown in FIG. 2. It includes broadband light source 10, which may be a xenon lamp, white light LED, or different color LEDs. The light passes through lens 12 and heat absorbing glass 14, and then passes through aperture 16. The light next passes through a first optical diffuser 18, tunable filter 20 (which may be a liquid crystal tunable filter or rotational mechanical filter) and a second optical diffuser 18. After exiting second optical diffuser 18 the light goes to ring light guide 22 which illuminates the tissue (not shown). Light reflected from the tissue passes through telecentric lens 40 and reaches camera 30, both of which are attached to articulated arm 60. Camera 30 may be a CCD or CMOS camera. Cables transmit data from tunable filter 20 and camera 30 to processor 50, which can be a personal computer or laptop. Alternatively data can be transmitted wirelessly. A laser-guided implement 70 is controlled by the processor. The laser-guided implement may be a pen which draws an outline on the tissue based on a signal transmitted by the processor, the outline being determined by the image stored in the processor. Alternatively, the laser-guided implement may be a cutting instrument such as a scalpel or a laser which cuts the tissue based on a signal transmitted by the processor. The signal transmitted by the processor may be transmitted wirelessly, or there may be a hard-wired connection.

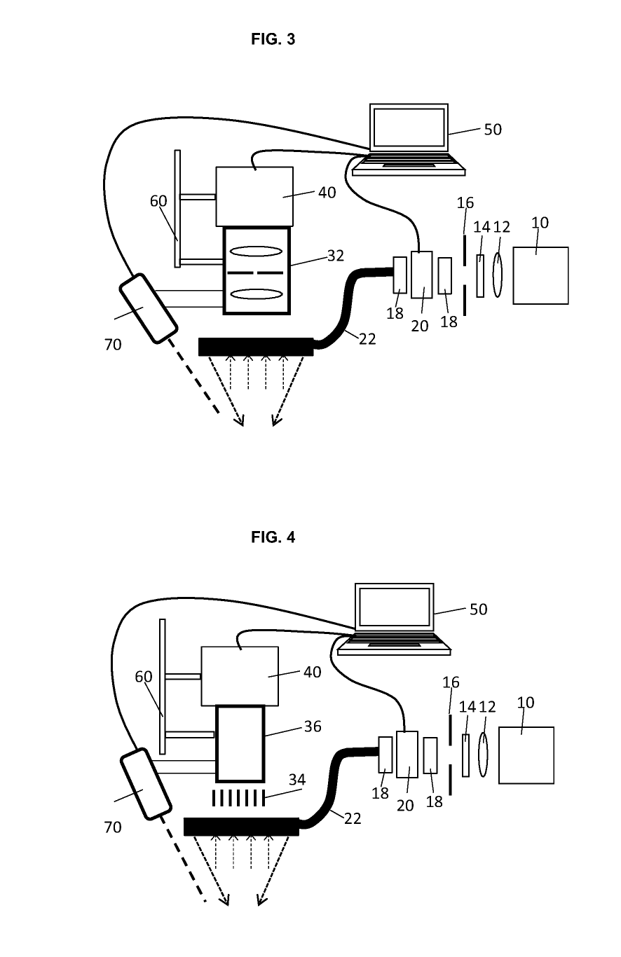

FIG. 3 shows an alternative embodiment of an optical imaging system of the invention for use in a clinical setting. The system in FIG. 3 is the same as the system in FIG. 2, except that back-directional gating component 32 is used instead of telecentric lens 40.

Another alternative embodiment of an optical imaging system of the invention for use in a clinical setting is shown in FIG. 4. The system in FIG. 4 is the same as the system in FIG. 2, except that anti-scatter grid 34 and camera lens 36 are used instead telecentric lens 40. Anti-scatter grid 34 may be a parallel grid, honeycomb grid, or mesh.

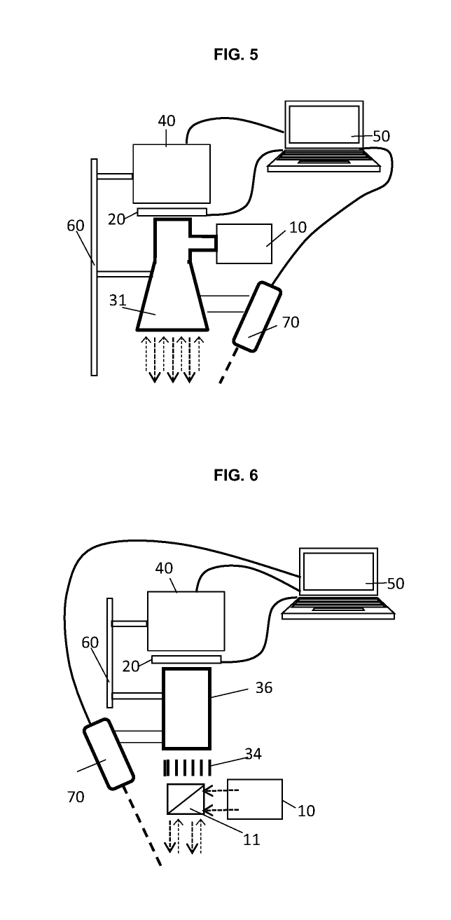

FIG. 5 shows yet another embodiment of an optical imaging system of the invention for use in a clinical setting. It includes broadband light source 10, which may be a xenon lamp, white light LED, or different color LEDs. Light travels to in-line illumination telecentric lens 31 and illuminates a tissue (not shown). Light reflected from the tissue passes back through in-line illumination telecentric lens 40, and then passes through tunable filter to camera 30. Cables transmit data from tunable filter 20 and camera 30 to processor 50, which can be a personal computer or laptop. Alternatively data can be transmitted wirelessly. A laser-guided implement 70 is controlled by the processor. The laser-guided implement may be a pen which draws an outline on the tissue based on a signal transmitted by the processor, the outline being determined by the image stored in the processor. Alternatively, the laser-guided implement may be a cutting instrument such as a scalpel or a laser which cuts the tissue based on a signal transmitted by the processor. The signal transmitted by the processor may be transmitted wirelessly, or there may be a hard-wired connection.

A further embodiment of an optical imaging system of the invention for use in a clinical setting is shown in FIG. 6. The system in FIG. 6 is the same as the system in FIG. 5, except that anti-scatter grid 34 and camera lens 36 are used instead of inline telecentric lens 31.

In all of the embodiments, the processor 50 sends trigger signals to change the wavelength of tunable filter 20 and then acquires data from camera 40 at that wavelength. Processor 50 thus synchronizes tunable filter 20 and camera 40.

### Example 1

### Anisotropy Factor-Weighted Imaging for Assessing Tumor Microenvironments and Demarcation—Variation in Back-Directional Angle

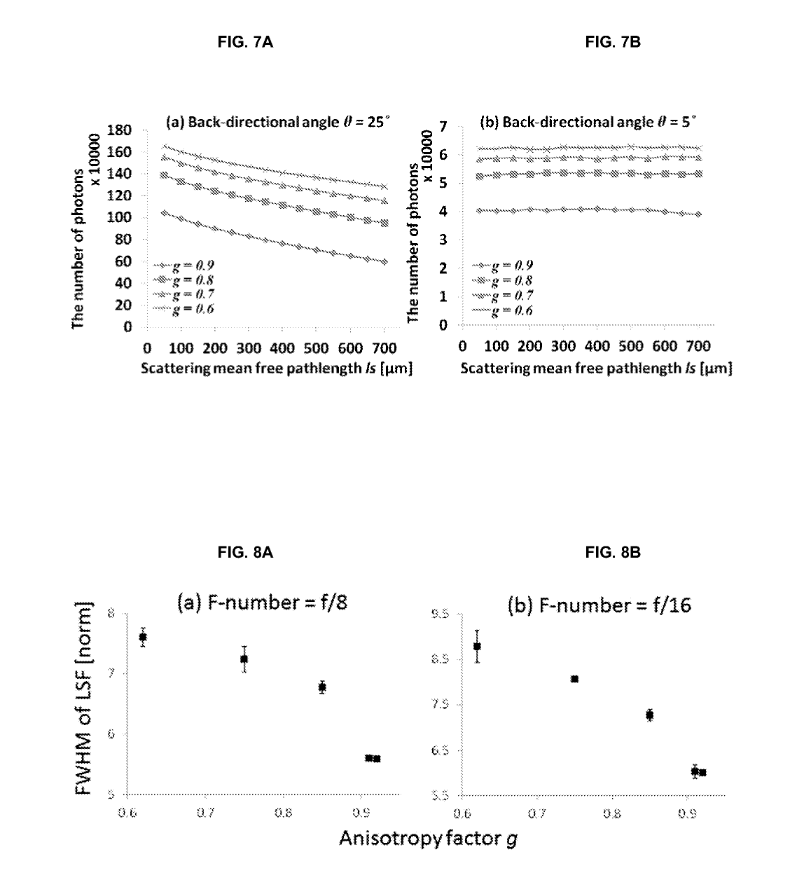

FIGS. 7A and 7B show the results of numerical experiments using optical ray-tracing combined with Monte-Carlo simulations. The number of photons detected by the virtual detector over different optical properties when the back-directional angle θ=25° (a) and θ=5° (b), corresponding to conventional imaging and back-directional gating, e.g., via a telecentric lens, setups, respectively. Surprisingly, when θ=5°, the number of collected photons does not depend on the scattering mean free path length Is for a given anisotropy factor g. Thus, the scattering intensity image obtained under back-directional gating can mainly be sensitive to changes in the anisotropy factor.

Using back-directional gating, e.g., via a telecentric lens, the number of collected photons does not depend on the scattering mean free path length. e.g., thickness of specimens, for a given anisotropy factor. In other words, at a given scattering mean free path length, the total hit of backscattered photons is mainly determined by changes in the anisotropy factor. These results show that back-directional gating allows the use of g as an imaging contrast. Importantly, as the anisotropy factor increases, the difference in the signal level becomes more significant. A small change at a high level of the anisotropy factor can produce a drastic change in the contrast. Given that most biological tissue is highly anisotropic, this indicates the ability to visualize a subtle change in the anisotropy factor induced by alterations in tissue structures and organizations. Thus, the scattering intensity or image obtained under back-directional gating is referred to as an anisotropy factor-weighted signal or image.

### Example 2

### Anisotropy Factor-Weighted Imaging for Assessing Tumor Microenvironments and Demarcation—Variation in F-Number

The full width at its half maximum (FWHM) of the line spread function (LSF) are obtained using a telecentric lens from a contrast target embedded in different anisotropic media. Results are shown in FIGS. 8A and 8B. The scattering media with different anisotropy factors (under the identical optical thickness of 5) are placed on top of a contrast target. The FWHM of LSF is normalized by the FWHM of LSF without the scattering media on top. The FWHM of LSF quantifies the resolving power, i.e., sample resolution, of the bottom layer, i.e., target, in the presence of the thick top scattering medium. The resolving power of the contrast target strongly depends on the anisotropy factor of the surrounding medium in the moderate depth. The error bars represent the standard deviation.

The resolving power of the contrast target is improved as the anisotropy factor increases. More interestingly, when the anisotropy factor reached to 0.8-0.95, the resolving power significantly increases. This result demonstrates that the image formation by a telecentric lens (i.e. back-directional gating) can effectively improve the resolving power of an embedded object when the surrounding scattering medium is highly anisotropic. Because in low anisotropic media, light is uniformly scattered to all directions, the back-directional gating does not isolate the ballistic or snake-like light. On the other hand, high anisotropic surrounding media in directional gating can serve as a waveguide to deliver the incident light to the embedded target and to isolate the ballistic or snake light scattered from the target.

### Example 3

### Analysis of Basal Cell Carcinoma Tissues Obtained from In Vivo Mohs Micrographic Surgery

Representative cases of resected thick tissue blocks (thickness=2-3 mm) with basal cell carcinomas obtained from Mohs micrographic surgery were analyzed. These were selected because it is well documented that basal cell carcinomas involve degeneration and realignment of the extracellular matrix. Four representative specimens with tumors that were confirmed histologically from adjacent slide sections were obtained. Case 1 was a nodular infiltrative basal cell carcinoma, Case 2 was a superficial nodular basal cell carcinoma, Case 3 was an infiltrative basal cell carcinoma, and Case 4 was a superficial nodular basal cell carcinoma. The fresh resected tissue blocks were imaged from the dermal side because residual tumors were included on the dermal side. The backscattering spectrum within λ=400 nm-700 nm at each (x, y) pixel was recorded using the back-directional gated spectroscopic imaging system of FIG. 1.

Absorption of hemoglobin in the visible spectral range can potentially mask the scattering contrast from the anisotropic effect. Unless hemoglobin absorption is removed, low intensity can be attributable to both strong hemoglobin absorption and high anisotropicity. To exclude the absorption from hemoglobin, a model comprised of power-law dependence on the wavelength A for the scattering component and Beer's law for the absorption of hemoglobin including a packaging effect correction were used. After fitting each spectrum at each (x, y) pixel to the model, the scattering contribution, i.e., power-law decay over λ, was extracted and a scattering intensity map deprived of the hemoglobin absorption was generated. Two anisotropy factor-weighted images were generated by summing the intensity within λ=400-450 nm and λ=650-700 nm for each specimen after the non-uniformly distributed hemoglobin contribution was removed.

Pseudo-color images were generated within the longer (λ=650-700 nm) and shorter (λ=400-450 nm) wavelength ranges, respectively. In order to obtain objective tumor contrasts accounting for specimen-to-specimen variations and to compare two different anisotropy factor-weighted images from the same specimen, the intensity of images was normalized to the same scale. Histology from the most adjacent thin frozen section (thickness=5 μm) also was analyzed. Each histological image was generated by mosaicing approximately 20 low-resolution microscopy images from the adjacent thin slide section and the locations of basal cell carcinomas were identified and studied. Corresponding histology images of each specimen were generated by mosaicing approximately 20 microscopic images from the adjacent thin slide section. Basal cell carcinomas were confirmed from individual histological readings and were identified on the mosaic image by a Mohs surgeon who also served as a pathologist. In anisotropy factor-weighted images, the overall intensity within the tumors was lower than that of the surrounding tissue, suggesting that the localized basal cell carcinomas had highly anisotropic properties.

The shorter wavelength anisotropy factor-weighted images depicted fine spatial patterns in greater detail and displayed higher tumor-to-surrounding tissue contrasts. For example, the anisotropy factor-weighted image of Case 1 showed that the tumor region had a much lower intensity than that of the surrounding tissue, yielding a sharper border. The scattering intensity exponentially decayed over the wavelength, which enhanced the intensity at the lower wavelengths. In other words, at the lower wavelengths, the value of anisotropy factor-weighted image was significantly higher than at the longer wavelengths. In addition, a reduced optical thickness of the specimen at the longer wavelengths could potentially lead to more leakage to the transmitted light. Overall, anisotropy factor-weighted images at the shorter wavelengths depicted enhanced tumor contrasts as well as heterogeneous tissue organizations in detail. The tumor areas in the anisotropy factor weighted images were larger than the marked tumor regions in the histological images. For example, in Case 2 an additional low intensity area on the far right side of the specimen was visible. Given the relatively deep imaging depth of back-directional gating (˜0.5-2 mm), the larger tumor area in the anisotropy factor-weighted image indicated that the residual tumor in the tissue block was larger than that of the thin histological section. This is in part because the depth of anisotropy factor-weighted imaging is much deeper than the thin section of the histology slide.

### Example 4

### Microvascular Imaging to Predict Tumor Sites and Identify Areas at High Risk for Tumor Development

Twenty-five SKH-1 hairless albino mice were used for photocarcinogenesis experiments as they lack pigmentation that can induce variability in UVB-induced changes and do not require shaving or depilatory chemicals that can induce non-specific inflammation. Mice were irradiated with one minimal erythema dose of UVB (2240 J/m²) three times per week. This treatment consistently results in initial tumor formation within 11-12 weeks of treatment. Therefore UVB treatments were discontinues after 10 weeks of treatment. This resulted in a cumulative UVB dose of 67.2 kJ/m², which exceeds a known carcinogenic cumulative UVB dose of 26.2 kJ/m2. For celecoxib, i.e. COX-2 inhibitor, studies, a topical dose of celecoxib that has previously been shown to be effective in inhibiting photocarcinogenesis in SKH-1 mice was used. In this case, 0.5 mg of celecoxib in 0.2 ml acetone was applied immediately after each UVB irradiation and 3 times per week after discontinuation of the UVB irradiations. Control mice received acetone (vehicle) treatment alone. The irradiated mouse dorsal skin was imaged on a biweekly basis. To obtain sequential images from the identical areas over time, reference markers were placed on each mouse by tattooing small areas (<˜0.5 mm in diameter).

In biological tissue, Hgb is the primary absorber in the wavelength range of λ=400-600 nm. In this range, both oxygenated Hgb (oxyHgb) and deoxygenated Hgb (deoxyHgb) have unique absorption spectral patterns, while absorption from other major absorbance contents such as melanin and bilirubin are minimal. For in vivo imaging of animals, it is difficult to obtain the spectral shape only from the scattering contribution in the absence of any absorbers. However, when broadband absorption patterns are applied to assess Hgb absorption content, light scattering spectra can be modeled as a monotonous declining function of the wavelength such as an exponential decay or a power law decay, as shown in previous spectroscopic analyses, e.g., point measurement over a large volume of tissue. For example, the spectrum can be fitted to the following expression:

$$I = ( {c}_{0} {λ}^{- {c}_{1}} ) {e}^{- ( {C}_{{HbO}_{2}} {ε}_{{HbO}_{2}} + {C}_{Hb} {ε}_{Hb} )} \qquad ( 1 )$$

where C_{HbO}_{₂} and C_{Hb} are the concentrations of oxyHgb and deoxyHgb multiplied by the pathlength of light in the medium, ε_{HbO}_{₂} and ε_{Hb} are the wavelength dependent absorption coefficients of oxyHgb and deoxyHgb, respectively, and c₀ describes the overall intensity of the spectrum and c₁ is the scatter power.

Total Hgb concentration can be estimated from C_{HbO}_{₂}+C_{Hb}. The algorithms in the previous spectroscopic technologies would not be appropriate for our microvascular imaging, due to the significant data size generated by our imaging platform (e.g. ˜60,000 spectral analyses for one image). Thus, a simple pattern recognition method was used to assess the total Hgb level in each unit (x, y) position, based on the approximation that the scattering contribution from the tissue can be modeled as a smooth decay function of the wavelength. The scattering contribution from the tissue was estimated as a smooth polynomial function of the wavelength. Spectrum at each position of x and y is fitted to a second polynomial function in the range of 440-900 nm:

I_{model}(λ)=aλ²+bΔ+c (2)

where a, b, and c are fitting coefficients.

The absorption spectral area is defined as the summation of all data points in the difference between the model spectrum and the original spectrum within the spectral range 500 nm-625 nm. Within a typical range of tissue scattering properties, the absorption spectral area can serve as a relatively accurate quantification of the microvascular blood Hgb content at each pixel. We also validate the simple algorithm with the conventional spectral analysis using Equation (1). Due to reduced cross-talk among adjacent pixels derived from back-directional gating in high anisotropic media, our spectral analyses can provide accurate absorption levels attributed to Hgb content at individual pixels.

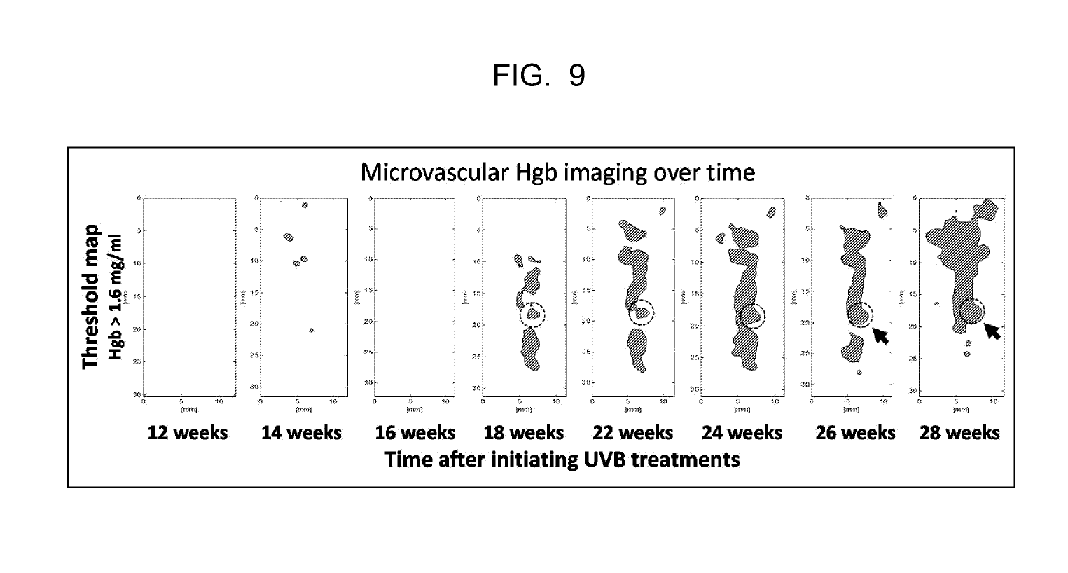

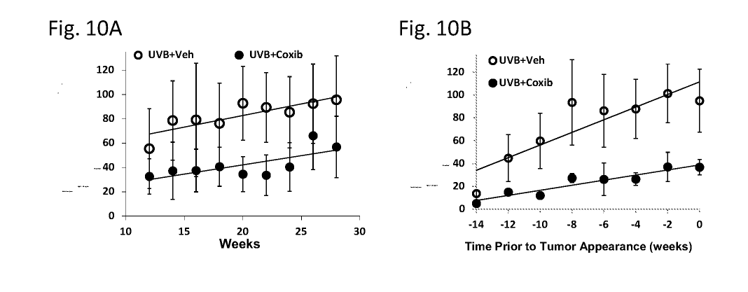

FIGS. 9 and 10 show expanding areas of high Hgb content following cessation of carcinogenic UVB treatment and celecoxib-resistant hyperemic foci exhibit a decrease in the area of high hyperemia.

FIG. 9 shows the UVB-induced hyperemic foci. Microvascular Hgb maps at biweekly intervals reveal spatial and temporal extent of focal hyperemia that persisted as well as expanded, leading up to tumor formation, after stopping UVB irradiations. Two arrows indicate the site of a macroscopic tumor that became visible in week 26 and 28.

FIG. 10A is a graph demonstrating that celecoxib blocked the formation and expansion of the areas of focally increased Hgb content, while the areas of high Hgb content continued to expand after cessation of UVB treatment in UVB treated mice (p=0.005 for the slope estimate over time in UVB+vehicle animals and p=0.029 in UVB+celecoxib mice). The slope estimates between the two groups were statistically the same with each other. The overall difference of focal hyperemic area between the two groups was statistically significant (p=0.044).

FIG. 10B shows that the appearance of tumors was preceded by expanding areas of high Hgb content in the weeks preceding tumor appearance. After a first tumor was observed in an imaging area, the area of high Hgb content in which the tumor appeared was calculated in the preceding microvascular Hgb images. The slope estimates of the linear regression for both groups were significant (p=0.004 for UVB+vehicle and p=0 for UVB+celecoxib). The difference in the slopes between the two groups was statistically significant (p=0.021).

The results showed that focal areas of persistent subclinical hyperemia were predictive of future tumor occurrence and showed celecoxib's chemopreventive ability. For UVB studies, it was desired to avoid the influence of UVB-induced acute inflammation (sunburn). Therefore a carcinogenic dose of UVB was administered over the first 10 weeks and then discontinued. This allowed examination of changes in regional blood supply prior to the time when tumors initially begin to appear in this mouse model, but after the acute sunburn effect had resolved. The effects of celecoxib on UVB-induced microvascular blood supply also were examined. Areas of focal hyperemia not only persisted, but expanded following cessation of carcinogenic UVB treatment. As noted above, after discontinuing UVB treatments, areas of increased hyperemia that persisted or formed over the ensuing weeks and months were noted. These persistent areas of focal hyperemia were seen to expand in area prior to tumor formation. This conclusion was verified by a plot of the imaged area that exhibited a threshold map of Hgb content >1.6 mg/ml. UVB-irradiated mice treated with vehicle showed a significant increase in area over time after stopping UVB irradiations. In contrast, celecoxib treated mice exhibited a reduction in the overall area of skin with high Hgb content at each imaging time point. Moreover, while celecoxib-resistant hyperemic foci also persisted, the rate of expansion (slope estimate over time) was not statistically different from that of UVB irradiated control (UVB+veh). To rule out the possibility that this expansion was simply reflecting the appearance of tumors (and tumor-associated angiogenesis), hyperemic areas that were present and persisted prior to the development of each visible tumor also were examined. An expanding area of high Hgb content that preceded the formation of all of the visible tumors in vehicle treated mice was demonstrated. Celecoxib treatment resulted in a significant decrease in the area of increased Hgb content. Moreover, celecoxib treatment reduced the rate of increase in hyperemic foci formation prior to overt tumor formation (the slope estimate of hyperemic area over time in UVB+coxib was statistically lower than that in UVB+Veh). These spatio-temporal observations are striking evidence that subclinical subepithelial inflammatory angiogenic foci occur prior to overt tumor formation, that these foci persist long after carcinogenic exposures cease, that these areas are highly predictive for future tumor formation, that celecoxib's ability to suppress tumorigenesis is tightly linked to its ability to reduce the area of subclinical inflammatory foci, and that celecoxib-resistant areas of inflammatory angiogenesis exhibit minimal changes in Hgb content, angiogenesis or tumor formation.

In addition to the telecentric arrangement disclosed herein, a simple yet effective image-based guidance method and system for treatment is also disclosed that are configured to provide projection display of histopathological information on a treatment field, which otherwise would be grossly normal-appearing tissue to clinicians. Although there are several recent advances and breakthroughs in biomedical imaging, image-guided procedures are still performed by displaying a contrast image overlaid with a background image on a monitor. As a result, there is an unmet need for an image-projection based guidance system that allows for imaging a clinically relevant area without relying on toxic contrast agents and for projecting a display map of the spatial extent of important biological alterations on the treatment field in real-time. The method and system disclosed herein combine label-free imaging of tissue/tumor microenvironments and optical projection to digitally display a mesoscopic map on the treatment surface in real-time or nearly real-time. In addition, the co-axial configuration of the ‘telecentric’ arrangement provides the ability to easily co-register an acquired/processed image to the same treatment site on a patient. Hence, the optical arrangement discussed herein can be incorporated into an instrument of modest price, increasing the rapid clinical adoption.

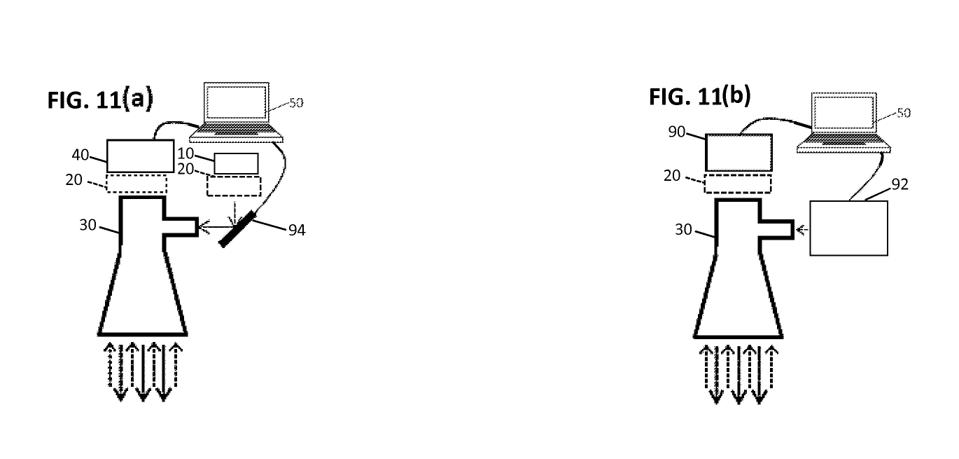

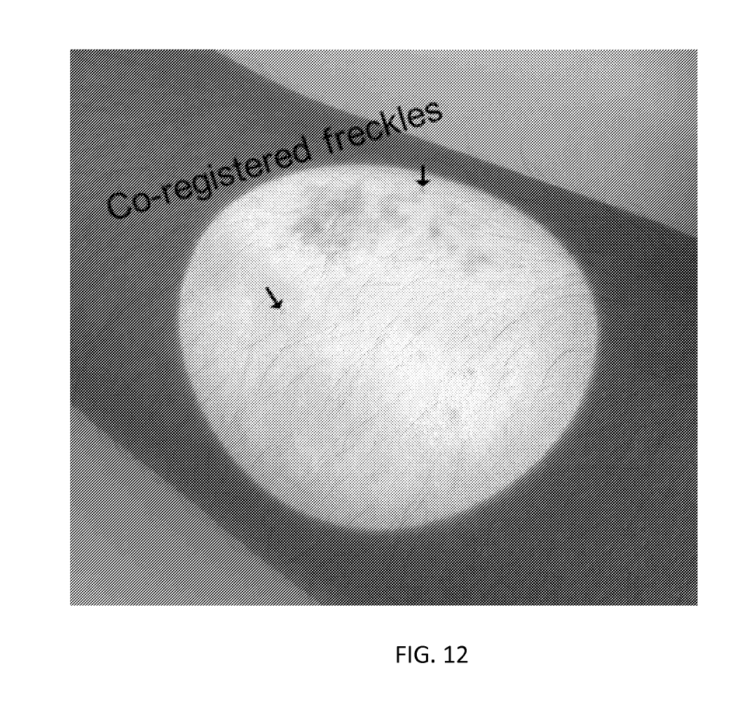

Referring to FIGS. 11(a)-11(b), various embodiment of a co-axial configuration which delivers spatially uniform illumination onto the tissue site for imaging and to couple a projection beam onto the same site for display projection for image co-registration are depicted. Via tunable wavelength filters (e.g., a liquid crystal tunable filter (LCTF), mechanical filter wheel, imaging spectrograph) 20 and a digital micromirror device (DMD) 94, a white-light source 10 is coupled to the light delivery port for co-axial illumination on the side. The light reflected from the tissue is collected by the same telecentric lens 30 at a distance (non-contact mode) and is coupled to a color camera of a charged coupled device (CCD) variety 40 or of a complementary metal oxide semiconductor (CMOS) 90 variety. The in-line telecentric lens 30 can be mounted on a flexible articulated arm (not shown) to have a relatively compact system for easy maneuvering and operation. Imaging and projection can be alternated as follows: 1) During image acquisition, DMD 94 is turned off and the wavelength of tunable wavelength filters 20 is varied from 400 nm to 1400 nm (i.e. visible and near infrared ranges). This mirror mode allows acquiring a data set of (x, y, and wavelength λ) for spectral analyses. 2) For display projection, a single-color illumination (e.g. green) from a white light source (e.g., LED, xenon lamp, deuterium lamp) 10 can be selected using the tunable wavelength filters 20 and the acquired/processed image is then provided to DMD 94 which then provides the image to the telecentric lens 30 in the side port which can then be projected onto the tissue. An optimal visual contrast of the projected image can also be determined, considering typical skin color. In FIGS. 11(a)-11(b), the tunable wavelength filters 20 are shown in dashed lines to indicate optional (i.e., in the image capture path, the tunable wavelength filters 20 may be used as discussed earlier, e.g., FIGS. 1 and 5, and in the projection path (FIG. 11(a)) to denote an option of providing a single-color illumination vs. providing white-light). Alternatively, a projector 92 can be used instead of the DMD 94 to provide the desired image to the telecentric lens 30.

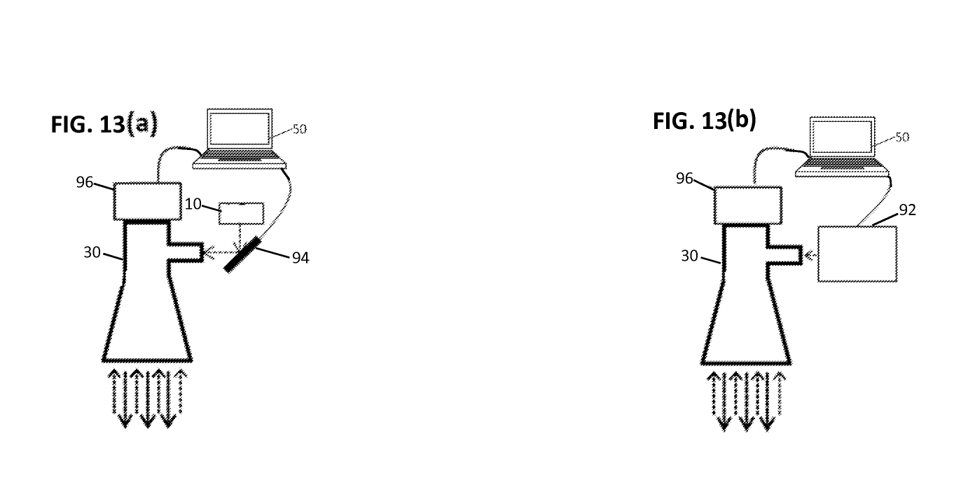

The arrangements shown in FIGS. 11(a)-11(b) may use tunable wavelength filters 20 which limit the development of fast, compact, and cost-effective systems. In particular, the image acquisition of the spectral data obtained from tunable wavelength filters 20, which also increases the physical dimension of the system and the cost of the system, can be a major limiting factor to achieve a (nearly) real-time system. Since it is advantageous to decouple the scattering and absorption contributions from the tissue, an automatic wavelength selection filter (e.g., mechanical filter wheel, imaging spectrograph, a liquid crystal tunable filter (LCTF)) would be necessary to obtain detailed spectral information. As illustrated in FIGS. 13(a)-13(b), a non-spectroscopic system can be realized by utilizing an algorithm that can reliably reconstruct full spectral information from red-green-blue (RGB) image data of a 3-color camera 96, without using an automatic wavelength selection filter. Several methods for spectral reconstruction with RGB data are known. While, the spectral information acquired by color CCD/CMOS cameras is limited, the reconstruction of multispectral images from RGB data has been successfully shown using several different methods. The most common methods can be categorized into three groups: Wiener estimation, principal component analysis (PCA)-based linear models, and multivariate polynomial regression. Thus, these hyperspectral reconstruction methods using RGB data can result in a real-time imaging and projection without using a bulky and expensive spectroscopic component.

For biological tissue imaging, multivariate polynomial regression works optimally among the different reconstruction methods. A hyperspectral reconstruction algorithm that can reliably predict detailed spectral information from RGB data can be provided as follows:

First, a reconstruction model is built using a group of training data set (i.e. training stage). To build a model for reconstructing reflectance spectra from RGB data, an RGB camera response v is expressed such that

v=M×r,

where r is the reflectance spectrum, and
M is the spectral response of the system. M can be obtained from the manufacturer of the three-color camera 96 or measured manually. Because M is the reference signal measured by a reflectance standard, M corresponded to all of the system responses, including the light source, the fiber optic light guide, the telecentric lens, and the spectral sensitivity of the three-color camera 96.

Second, r is expressed to estimate r given M and v in terms of a conversion matrix T such that r=v×T.

Third, T is determined by applying multivariate polynomial regression.

Finally, r is computed. In this step, an optimal order of polynomial degree can be determined to provide highly reliable performance for the reconstructed spectrum r. The reconstruction accuracy can be evaluated using goodness-of-fit metrics or root mean square errors and the predictive ability can be tested using cross validations (e.g., leave-one-out, known to a person having ordinary skill in the art).

Thus, a mesoscopic imaging apparatus and method have been described according to the present invention. Many modifications and variations may be made to the techniques and structures described and illustrated herein without departing from the spirit and scope of the invention. Accordingly, it should be understood that the methods and apparatus described herein are illustrative only and are not limiting upon the scope of the invention.

1. A mesoscopic imaging and surgical guidance apparatus, comprising:
an image capturing path, including:
a telecentric lens which receives backscattered light from a light which illuminates tissue in vivo with diffused light or collimated light and transforms the received light into an orthographic view,
an image capturing device which captures the orthographic view,
a processing unit configured to process the captured orthographic view to thereby generate a guidance image; and
a projection path, including
a light source configured to project the generated guidance image through the telecentric lens on to the tissue to guide surgical operations.

2. The mesoscopic imaging and surgical guidance apparatus of claim 1, further comprising:
a tunable wavelength filter disposed in the image capturing path.

3. The mesoscopic imaging and surgical guidance apparatus of claim 1, wherein the tunable wavelength filter is disposed between the telecentric lens and the image capturing device.

4. The mesoscopic imaging and surgical guidance apparatus of claim 2, wherein the image capturing device is one of a charged coupled device (CCD) digital camera and a complementary metal oxide semiconductor (CMOS) digital camera.

5. The mesoscopic imaging and surgical guidance apparatus of claim 1, the processor is one of a personal computer, an application specific integrated circuit, a microcontroller, and a combination thereof.

6. The mesoscopic imaging and surgical guidance apparatus of claim 1, further comprising:
a tunable wavelength filter disposed in the projection path.

7. The mesoscopic imaging and surgical guidance apparatus of claim 6, wherein the tunable wavelength filter disposed between the light source and the telecentric lens, and configured to generate a single-color illumination.

8. The mesoscopic imaging and surgical guidance apparatus of claim 1, further comprising:
a digital micromirror device disposed in the projection path between the light source and the telecentric lens.

9. The mesoscopic imaging and surgical guidance apparatus of claim 1, wherein the light source is a projector.

10. A mesoscopic imaging and surgical guidance apparatus, comprising:
an image capturing path, including:
a telecentric lens which receives backscattered light from a light which illuminates tissue in vivo with diffused light or collimated light and transforms the received light into an orthographic view,
a red-green-blue (RGB) digital camera which captures and provides the orthographic view in wavelengths associated with red, green, and blue (RGB image data),
a processing unit configured to process the captured RGB orthographic view to thereby generate a guidance image; and
a projection path, including
a light source configured to project the generated guidance image through the telecentric lens on to the tissue to guide surgical operations.

11. The mesoscopic imaging and surgical guidance apparatus of claim 10, wherein the light source is a projector.

12. The mesoscopic imaging and surgical guidance apparatus of claim 10, the processor is configured to:
(i) reconstructing full spectral information from the captured RGB image data using any one of Wiener estimation, principal component analysis (PCA)-based linear models, multivariate polynomial regression, and combinations thereof, and
(ii) generating real-time imaging.

13. The mesoscopic imaging and surgical guidance apparatus of claim 12, the reconstructing step includes:
(i) providing a group of training data set,
(ii) building a reconstructing model for reconstructing reflectance spectra from RGB image data, and
(iii) generating a RGB response, v, where v is determined by v=M×r, where r is the reflectance spectrum and M is the spectral response, where M is a predetermined constant, and r is determined by r=v×T, where T is a conversion matrix.
